(19) United States
(12) Plant Patent Application Publication
(10) Pub. No.: US 2017/0020044 P1
(43) Pub. Date: Jan. 19, 2017

(54) Apple tree named 'Berica'

(50) ;

(71) Applicant: Fashion Foods Limited, Nelson (NZ)

(72) Inventor: William John Edmund LYNCH, Richmond (NZ)

(21) Appl. No.: 14/999,876

(22) Filed: Jul. 13, 2016

(30) Foreign Application Priority Data
Jul. 15, 2015 (NZ) APP232

(51) Int. Cl. A01H 5/00

(52) U.S. Cl. PLT/162

(57) ABSTRACT

A new and distinct variety of Malus domestica named ‘Berica’ characterized by a bright pink colour with faint underline stripe fully filling in to block colour; more intensively visually apparent bright pink tones than any comparable early maturing apple varieties; foreground colour over 75% of fruit surface; pale lemon background colour; random ‘flecking’ exhibited to varying modest degrees in approximately 30% of fruits; early maturation; distinctive strong flavours and after-tastes; consistent and stable repeat cropping returning identical characteristics over an extended trial period; and retention and enhancement of bright colouration in long storage events.

### GENUS AND SPECIES PLANT CLAIMED

Malus domestica

### VARIETY DENOMINATION

‘Berica’

### CROSS REFERENCE TO RELATED APPLICATION

This application claims the benefit of New Zealand Plant Variety Rights Application No. APP232, filed Jul. 15, 2015, the disclosure of which is incorporated by reference in its entirety.

### BACKGROUND OF THE INVENTION

The present invention relates to a new and distinct variety of apple tree hereinafter referred to as ‘Berica’.

The discovery was made in 1999 amongst a block of Heritage Gala apple trees growing on an orchard in the Tasman District of Nelson Province in New Zealand, whereby one tree on the end of a row of trees was identified that exhibited many and varied apparent mutations of the variety that was systematically planted in an orchard block of apple trees. Due to the location of the tree and its significant different attributes from all others, it was not possible to establish whether the tree was a seedling positioned strategically for expedient observation, or a chance mutation coincidentally occurring on the end of a row of trees, as the orchard was purchased with the trees established and enquiries failed to reveal any knowledge or information.

The inventor selected and propagated trees from the identified discovery to test and consider the attributes of the discovery, and after many years of observation, trials and elimination chose two principal new apple varieties that were distinctly different visually and in eating attributes.

‘Berica’ is one of the chosen selections and was propagated from the selected plant material and planted out on rootstocks M.793, M9, and CG202 in moderate numbers to further test and examine stability and identified characteristics.

### BRIEF DESCRIPTION OF THE INVENTION

Over a 15 year trial period, the inventor has established the following characteristics and differences that demonstrate ‘Berica’ as a new and distinct apple variety: 1) Bright pink with faint underline stripe fully filling in to block colour; 2) More intensively visually apparent bright pink tones than any comparable early maturing apple varieties; 3) Foreground colour over 75% of fruit surface; 4) Pale lemon background colour; 5) Random ‘flecking’ exhibited to varying modest degrees in approximately 30% of fruits; 6) Early maturation affording harvest 7 to 10 days in advance of regular ‘Gala’ and ‘Royal Gala’ varieties; 7) Distinctive strong flavours and after-tastes more intense than ‘Royal Gala’ and its mutations, and reminiscent of historical ‘Heritage Gala’; 8) Consistent and stable repeat cropping returning identical characteristics over an extended trial period; and 9) Retention and enhancement of bright colouration in long storage events, compared to fading and browning observed in regular ‘Royal Gala’ and ‘Gala’ strains when stored for extended periods.

Provided below are comparisons of the present variety to possible parental or other early maturing varieties.

| Characteristic | New Variety ‘Berica’ | Comparison variety |
|---|---|---|
| Hue over colour of | bright pink with | Heritage Gala orange/red |
| mature fruit | orange hint | Royal Gala-orange/red |
| Background colour at | mild cheddar | lemon |
| optimum harvest | | |
| Relative area of | 75%-95% | Heritage Gala 25%-50% |
| over-colour | | Royal Gala 66%-80% |
| Relative area of back- | 5%-25% | Heritage Gala 50%-75% |
| ground colour | | Royal Gala 25%-50% |
| Relative area of random | 0%-2.5% | Heritage Gala 0% |
| flecking | | Royal Gala 0% |
| Relative frequency of | 33.33% | Heritage Gala 0% |
| flecking (per 100 fruits) | | Royal Gala 0% |
| Relative eating | stronger flavour | Royal Gala noticeably |
| attributes | and after-tastes | lesser than traditional |
| | | Gala & Royal Gala |

### BRIEF DESCRIPTION OF THE DRAWINGS

The accompanying photographs illustrate the overall appearance of the new malus ‘Berica’ showing the colours as true as is reasonably possible with coloured reproductions of this type using a 44 mp camera.

FIG. 1 shows a perspective view of cropping ‘Berica’ trees with fruit immediately prior to harvest.

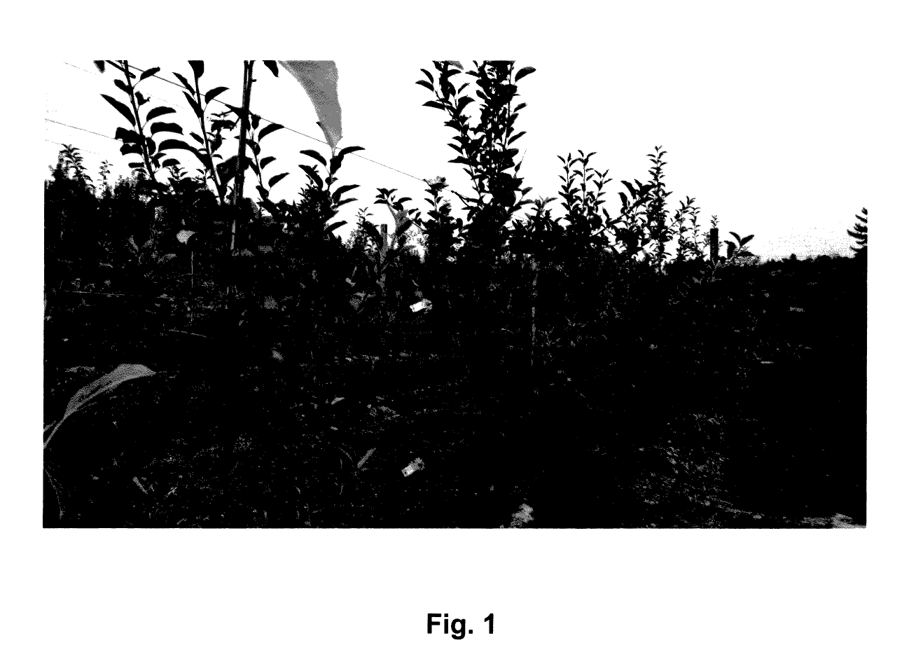

FIG. 2 shows a perspective view of the harvested ‘Berica’ fruit.

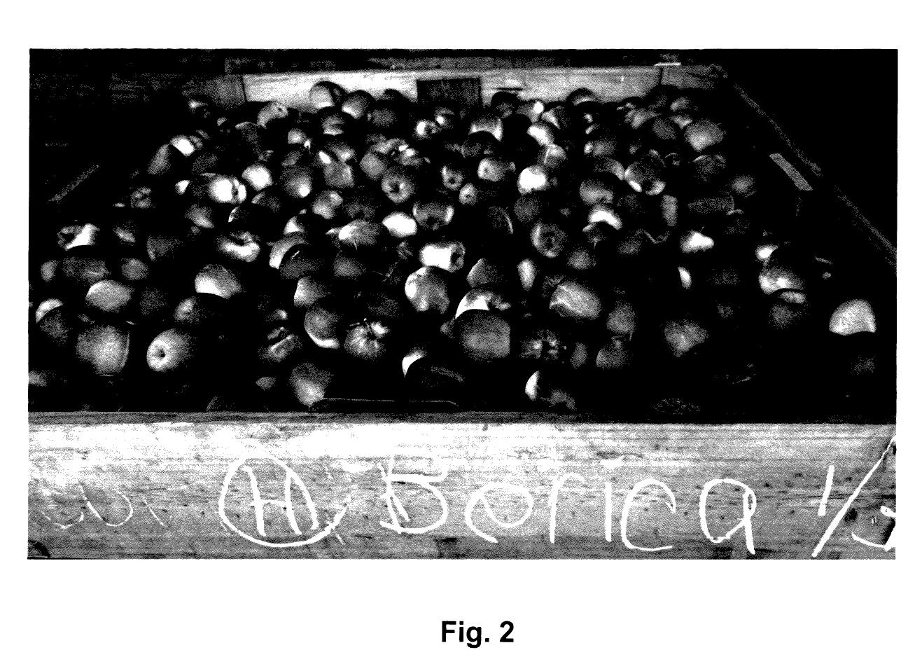

FIG. 3 shows a perspective comparison of harvested ‘Berica’ fruit alongside ‘Heritage Gala’ and ‘Royal Gala’ exhibiting distinctive visual differences.

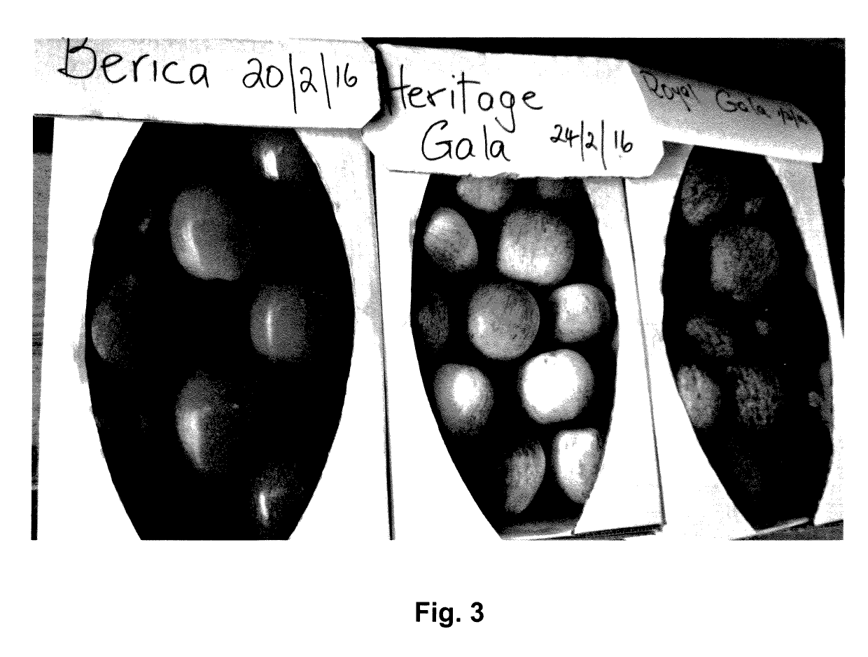

FIG. 4 shows ‘Berica’ random “flecking”.

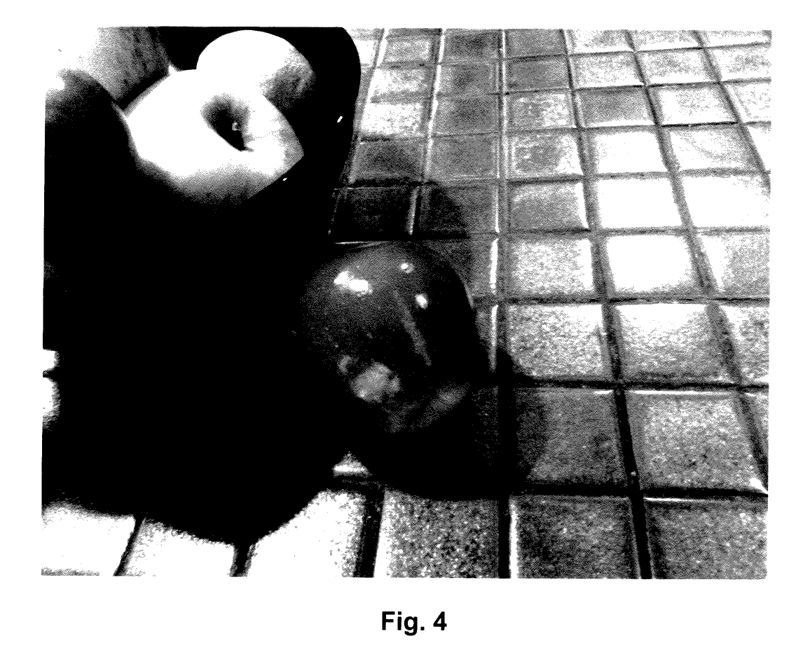

FIG. 5 shows mild cheddar background colour.

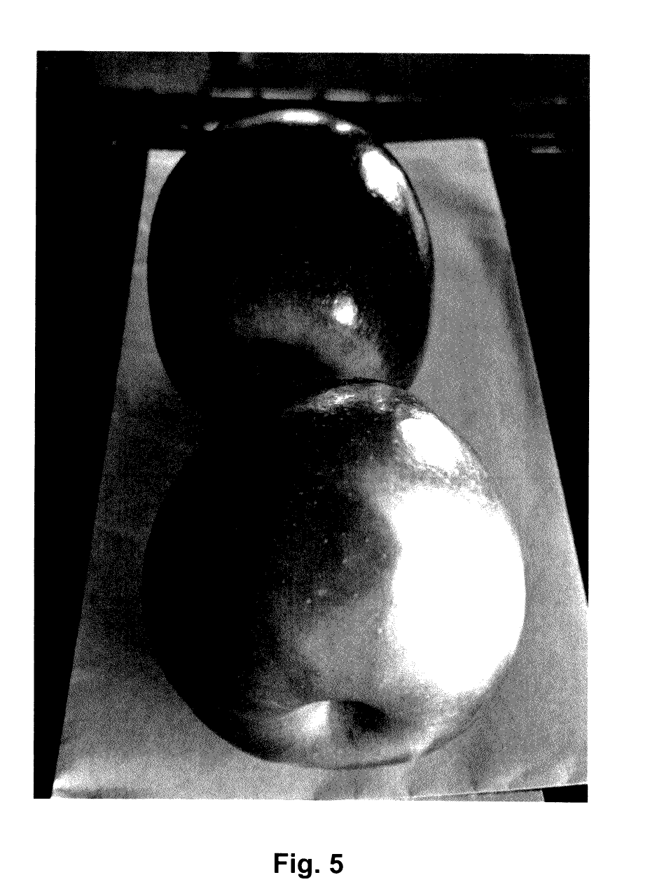

FIG. 6 shows vivid pink colouration extenuated by long periods of cool storage.

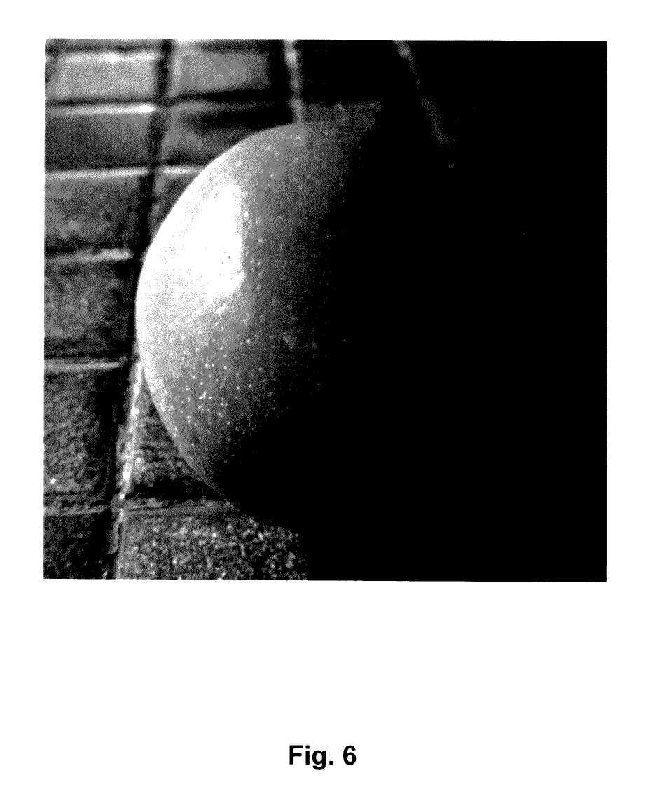

FIG. 7 shows the full colouration and maturity development of ‘Berica’ 10 days before ‘Royal Gala’ harvest.

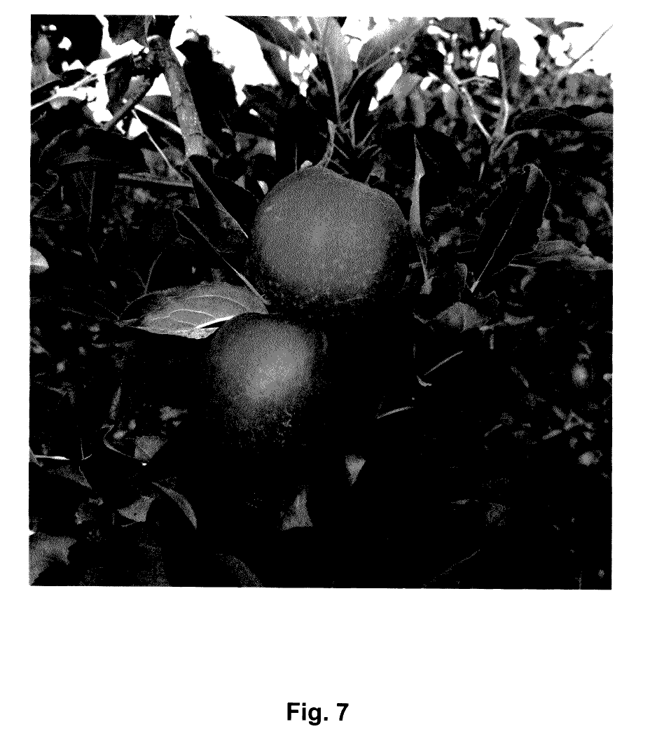

### DETAILED DESCRIPTION

The following is a detailed description of the new variety. The new malus variety ‘Berica’ has not been observed under all possible environmental conditions.

The aforementioned photographs together with the following observations and values, describe the trees of ‘Berica’ as grown in an orchard situated in Redwood Valley, Tasman District, Nelson Province of the South Island of New Zealand, Latitude S. 41, E2516661, N5989297, 36-50 m above sea level.

The planting site and climatic conditions closely approximate those generally used in commercial production of pipfruit, and the land areas in the vicinity have successfully been deployed in the production of pipfruit since the settlement of the Region, so that the location is generally viewed as a suitable site for commercial production.

The climate is in the temperate zone with summer temperatures generally below 30 deg C. and maximum 35 deg C. Winter temperatures range 7-16 deg with minimums minus 5-7 deg.

The soil is variable and of modest fertility, being a mix of recent alluvial soils with clay type sub-soil, with fertility maintained by applications of fertiliser applied annually.

Trees are irrigated by drip irrigation when necessary. Annual rainfall is 900-1200 mm per year.

The trial blocks are substantially planted on CG 202 rootstock with the first commercial planting trial block now in 6^{th} year. Plantings are spaced 1.5 M between tree and 3.5 m row width giving a tree density of 2000 trees per hectare. All trees have performed identically in respect of fruit characteristics.

The type of bearing is early, being the first harvestable variety. Productivity is good with regular and heavy flowering and no bi-annual flowering evident.

Production on mature trees is estimated to be 20 kg-30 kg per tree.

Average size of mature trees is 3 m height and spread 1.4 m.

Trunk calliper is around 5 cm (10 cm above grafting).

Trunk texture is smooth with numerous lenticels slightly erased.

Trunk bark colour brown-greyed RHS 197C and the lenticels are about 5 mm long×1.6 mm wide.

Branches number about 15 per tree and are angled generally 45-75 deg. Natural growth tendencies have been suppressed by tying pendant as part of tree training management.

Blooming time in Redwood Valley is from early October till 3rd week of October with staggered later heavy flowering on one year old wood.

Fruit maturity commences second week of February which is about 125 days after flowering.

The harvest window is from early-mid February until early March.

Keeping quality is good on the tree and in storage. In Coolstore at 0.5 deg C. trials with Smartfresh ™ have stored good quality outcomes to 280 days. Controlled atmosphere storage trials are yet to be conducted.

Fruits are conical-truncated with average size approx. 85 mm and weight around 240 gm.

Stem length is around 27 mm with diameter 2.7 mm.

Stem cavity depth is 10 mm and has some smooth russet on about 40% of the fruits.

Skin is smooth texture of medium thickness.

Firmness at harvest time 7.8 kg-9.4 kg.

Colour is around RHS 52A with background lemon 1D and the random “flecking” characteristic 158B.

Seeds per fruit about 6 and per locule 1-2. Length 7.7 mm-width 4.7 mm.

Propagation is from budding and grafting on GC202 (patented) rootstock.

Light intensities—full sunlight or slight shade. A small trial area using light-reflective cloth has been conducted with anticipated outcome of increased percentage of foreground colour. The trial area will be increased.

Pruning requirements are similar to heritage “GALA” , requiring removal of strong upright growth, and systematic renewal of fruiting wood. The trees perform well with branches trained into a pendant position of approximately 45% below horizontal. Flower numbers are very high and require heavy blossom thinning over the bloom period.

Tree vigour is moderate with uptight form and good ramification.

Main branches develop with flat angles around 45%-50% if not trained down.

Use is the fresh fruit market as a desert apple from March until December in NZ.

Disease/pest susceptibility is evident to black spot (scab), and powdery mildew at increased incidence requiring dedicated control.

Climatic hardiness appears normal tolerance for planted site which experiences summer temperatures in the mid to high 30's and winter temperatures down to −10 deg C.

I claim:

1. A new and distinct Malus domestica variety of apple tree named ‘Berica’ as illustrated and described herein
